(19) United States
(12) Patent Application Publication
(10) Pub. No.: US 2013/0012770 A1
(43) Pub. Date: Jan. 10, 2013

(54) COMBINED RETRACTOR AND ENDOSCOPE SYSTEM

(75) Inventor: YING-CHIEH SU, Tainan City (TW)

(73) Assignee: Chi Mei Medical Center, Tainan City (TW)

(21) Appl. No.: 13/539,771

(22) Filed: Jul. 2, 2012

(30) Foreign Application Priority Data
Jul. 6, 2011 (TW) 100123885

(51) Int. Cl. A61B 1/32; A61B 1/06; A61B 1/04

(52) U.S. Cl. 600/104

(57) ABSTRACT

A combined retractor and endoscope system includes a retraction ring and an endoscope unit. The retraction ring includes a tubular inner surface defining a passageway. The endoscope unit includes a flexible arm that can be positioned stably and moved between an edge of an incision and an outer peripheral surface of the retraction ring, and a camera attached to one end of the flexible arm. In a preferred embodiment, the flexible arm has a flexible outer tube body incorporating a support strip that can be bent together with the outer tube body to retain a bent configuration of the outer tube body.

### CROSS REFERENCE TO RELATED APPLICATION

This application claims priority of Taiwanese Application No. 100123885 filed on Jul. 6, 2011.

### BACKGROUND OF THE INVENTION

1. Field of the Invention

The invention relates to a surgical operation system, and more particularly to a combined retractor and endoscope system for use in laparoscopy or thoracoscopy surgery.

2. Description of the Related Art

It is well known to use a surgical retractor for retracting an incision in an abdominal wall and for exposure of an interior part of a patient's body during a laparoscopy or thoracoscopy operation. The commonly used retractors include manually and mechanically operated retractors, such as a plain retractor, a rake retractor, a self-retaining retractor, etc. Generally, conventional retractors require a relatively complicated retraction procedure in use, and are composed of metal parts that are likely to exert uneven pressure and cause injury to the sides of an incision and that may damage other surgical instruments upon frictioning and impacting during surgery.

An endoscope device is used when it is necessary to examine and visualize an internal body part of a patient during surgery. Usually, a surgeon will hold the endoscope device to insert it into a patient's body through an incision and manipulate it to conduct initial examination before surgical treatments. When other surgical instruments are to be manipulated for subsequent treatment steps, the endoscope device is transferred to and held by another medical personnel to assist in operating the endoscope device. The surgical procedure as such is inconvenient.

### SUMMARY OF THE INVENTION

One object of the present invention is to provide a combined retractor and endoscope system that is simple in structure and convenient and safe in use.

According to one aspect of the present invention, a combined retractor and endoscope system comprises a retraction ring and an endoscope unit. The retraction ring is adapted for insertion into and for retraction of an incision in a patient's body, and includes a tubular inner surface defining a passageway. The endoscope unit includes a flexible arm that can be positioned movably between an edge of the incision and an outer peripheral surface of the retraction ring, and a camera attached to one end of the flexible arm.

According to another aspect of the present invention, an endoscope for use in combination with a retraction ring, comprises: a flexible arm that is bendable to form a substantially U-shape and to have a bent part between two ends of the flexible arm; and a camera attached to one of the ends of the flexible arm. The bent part can be positioned movably between an edge of an incision and an outer peripheral surface of the retraction ring after the camera extends through the incision.

### BRIEF DESCRIPTION OF THE DRAWINGS

Other features and advantages of the present invention will become apparent in the following detailed description of the preferred embodiments with reference to the accompanying drawings, of which:

FIG. 1 is a schematic view illustrating the first preferred embodiment of a combined retractor and endoscope system according to the present invention;

FIG. 2 is a schematic view illustrating the first preferred embodiment in use; and

FIG. 3 is a perspective view of the second preferred embodiment of the combined retractor and endoscope system according to the present invention.

### DETAILED DESCRIPTION OF THE PREFERRED EMBODIMENTS

Before the present invention is described in greater detail with reference to the accompanying preferred embodiments, it should be noted herein that like elements are denoted by the same reference numerals throughout the disclosure.

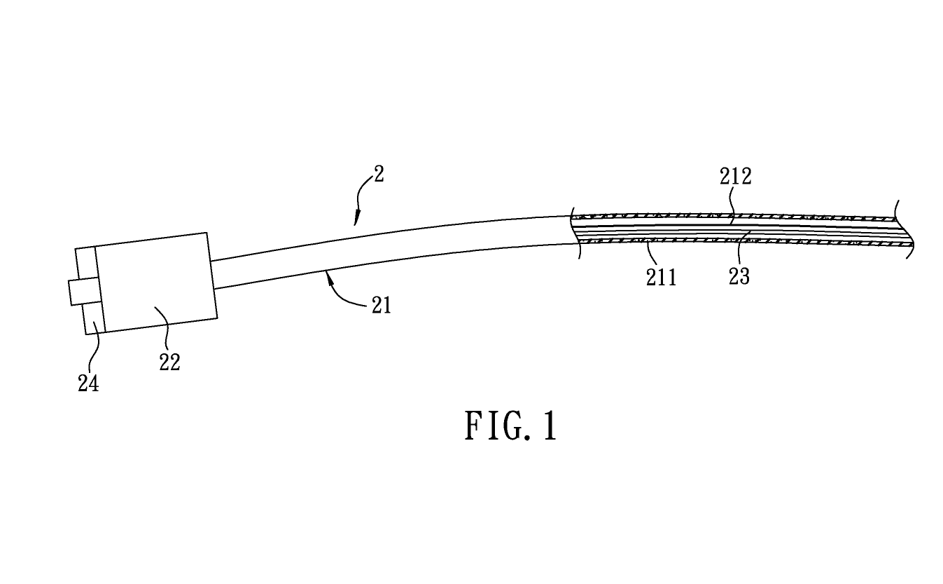

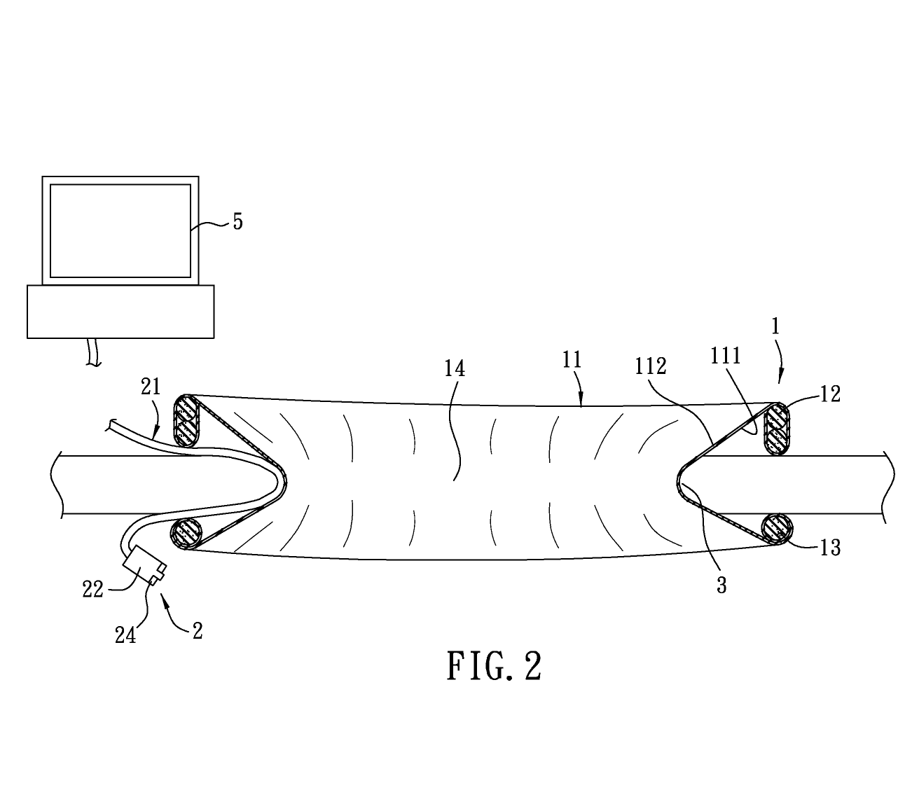

Referring to FIGS. 1 and 2, there is shown the first preferred embodiment of a combined retractor and endoscope system according to this invention, which may be used for laparoscopy, and which includes a retraction ring 1, and an endoscope unit 2.

The retraction ring 1 is adapted for insertion into and for retraction of an incision 3 in a patient's body, and is made of a resilient material. The retraction ring 1 can retract the incision 3 by applying uniform pressure to the edges of the incision 3 in all directions of 360 degrees to form a circular opening. The retraction ring 1 includes a tubular membrane 11 for retraction of the incision 3, a first ring 12 connected to an outer tubular end of the tubular membrane 11 to roll the outer tubular end, and a second ring 13 connected to an inner tubular end of the tubular membrane 11 to roll the inner tubular end. The tubular membrane 11 includes a tubular inner surface 112 defining a passageway 14. By folding and rolling the outer or inner tubular end of the tubular membrane 11 about the first or second ring 12, 13, the tubular membrane 11 can be stretched tightly to retract the incision 3 into a predetermined size and shape for exposure of the interior body part of the patient during surgery. In this embodiment, the first ring 12 are formed by two stacked plastic rings each having an oval cross section, and the second ring 13 is a single plastic ring having a circular cross section. The tubular membrane 11 is made of a rubber material.

The endoscope unit 2 includes a flexible arm 21 that can be positioned movably between the edges of the incision 3 and an outer surface 111 of the retraction ring 1, a camera 2 attached to one end of the flexible arm 21 for recording the conditions inside the patient's body, a transmission cable 23 extending through the flexible arm 21 and connected to the camera 22, and a light emitter 24 disposed at the end of the flexible arm 21. In this embodiment, the transmission cable 23 is a digital signal transmission cable, and the camera 22 is a charge coupled device (CCD). However, in practice, the camera 22 may also be a complementary metal-oxide semiconductor (CMOS).

While the light emitter 24 may be a light-emitting diode in this embodiment, it should not be limited so in practice. In addition, the light emitter 24 is not an absolutely essential component and may be dispensed with according to the present invention.

When the camera 22 acquires a video signal, the video signal will pass through the transmission cable 23 for subsequent output to a display device 5 connected to another end of the flexible arm 21 that is disposed outside the patient's body.

In this embodiment, the flexible arm 21 has a flexible outer tube body 211, and an elongate support in the form of a support strip 212 that is inserted into the flexible outer tube body 211 and that is bendable together with the flexible outer tube body 211 to retain a bent configuration of the flexible outer tube body 211. According to this embodiment, the outer tube body 211 may be made of silicone rubber, and the support strip 212 may be a bare metal strip, or a metal strip shielded by a plastic layer. The outer tube body 211 may have a flat-shape cross section, a shuttle-shaped cross section, or any other suitable non-circular cross section.

According to another preferred embodiment, the flexible arm 21 may include a metallic arm, and the transmission cable 23 may be a conductive layer formed on the metallic arm.

When the combined retractor and endoscope system is used for surgery, the retraction ring 1 is put into the incision 3 with the first ring 12 and the second ring 13 being disposed outside and inside the patient's body, respectively. A surgeon may grip the first ring 12, and turn it inside out, causing the outer tubular end of the tubular membrane 11 to fold and roll about the first ring 12. As a result, the tubular membrane 11 is stretched outward radially and tightly, retracting the incision 3 into a circular shape with a predetermined size. Since the diameters of the first ring 12 and the second ring 13 are larger than that of the incision 3, the tubular membrane 11 converges from the inner and outer tubular ends to its intermediate part contacting the incision 3. The flexible arm 21 of the endoscope unit 2 is bent into a U shape and is inserted in between the outer peripheral surface 111 of the tubular membrane 11 and the edges of the incision 3 . The bent part of the flexible arm 21 can be positioned between the outer peripheral surface 111 of the tubular membrane 11 and the edge of the incision 3 after the camera 22 extends through the incision 3. Because the bent part of the tubular membrane 11 abuts tightly against the edge of the incision 3 and because the flexible arm 21 can be stably retained in the U-shape, the flexible arm 21 can be firmly clamped and positioned between the edge of the incision 3 and the tubular retraction membrane 11 to allow a surgeon to easily manipulate the endoscope unit 2. The surgeon may hold and move the flexible arm 21 in an angular direction around the outer surface 111 of the tubular membrane 11 to acquire and send video signals to the display device 5. When the surgeon releases the flexible arm 21, the flexible arm 21 may be retained at a desired position.

Because the retraction ring 1 made of resilient material exerts a uniform pressure to the edges of the incision 3 to retract the tissue around the incision 3 and forms an annular membrane wall to contact intimately against the sides of the incision 3, the retraction ring 1 can protect the incision 3 from injury, infection and contamination. Besides, since the flexible arm 21 of the endoscope unit 2 can be positioned firmly between the outer peripheral surface 111 of the retraction ring 1 and the edge of the incision 3, the flexible arm 21 need not be positioned by hand or by any additional holding means.

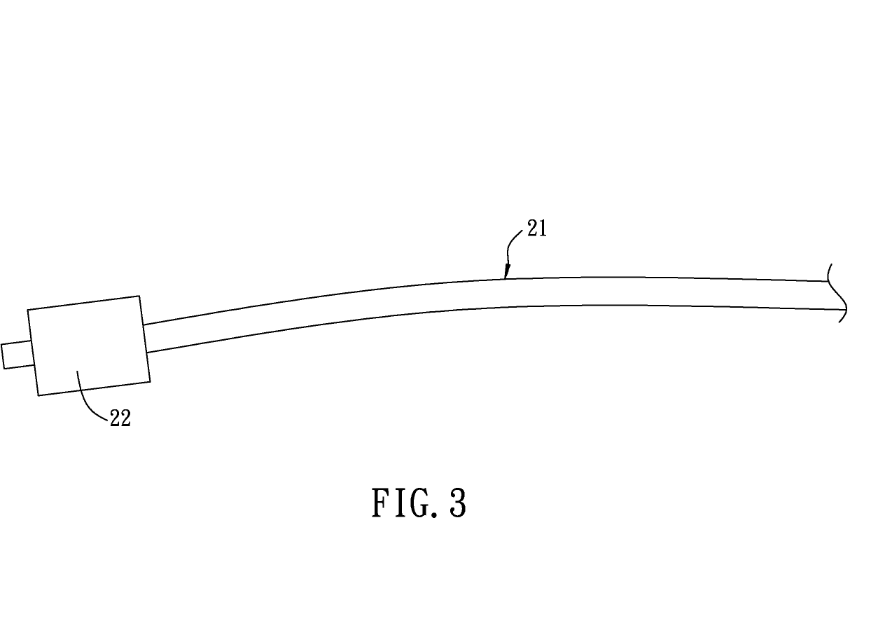

Referring to FIG. 3, the second preferred embodiment of this invention differs from the first preferred embodiment in that the transmission cable 23 is a digital signal transmission optical fiber that can be used as alight guide. Therefore, an external light source (not shown in the Figure) may be used, and the light emitted thereby may be guided by the transmission cable 23 into the incision 3 . The light source 24 of the first preferred embodiment can be dispensed with in the second preferred embodiment.

While the present invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A combined retractor and endoscope system comprising:
a retraction ring adapted for insertion into and for retraction of an incision in a patient's body, and including an inner surface defining a passageway; and
an endoscope unit including a flexible arm that can be positioned movably between an edge of the incision and an outer peripheral surface of said retraction ring, and a camera attached to one end of said flexible arm.

2. The combined retractor and endoscope system of claim 1, wherein said flexible arm has a flexible outer tube body, and an elongate support that is inserted into said flexible outer tube body and that can be bent together with said flexible outer tube body to retain a bent configuration of said flexible outer tube body.

3. The combined retractor and endoscope system of claim 2, wherein said elongate support is a strip made of metal.

4. The combined retractor and endoscope system of claim 2, wherein said endoscope unit further includes a transmission cable extending through said flexible outer tube body and connected to said camera.

5. The combined retractor and endoscope system of claim 4, wherein said transmission cable is a digital signal transmission cable.

6. The combined retractor and endoscope system of claim 1, wherein said endoscope unit further includes a light emitter disposed at said one end of said flexible arm.

7. The combined retractor and endoscope system of claim 1, wherein said retraction ring further includes a tubular membrane for retraction of the incision, a first ring connected to a tubular end of said tubular membrane, and a second ring connected to another tubular end of said tubular membrane.

8. The combined retractor and endoscope system of claim 2, wherein said flexible arm is bent to form a substantially U-shape and has a bent part that can be positioned between an outer peripheral surface of said tubular membrane and an edge of the incision.

9. An endoscope for use in combination with a retraction ring, comprising:
a flexible arm that is bendable to form a substantially U-shape and to have a bent part between two ends of said flexible arm; and
a camera attached to one of said ends of said flexible arm;
wherein said bent part can be positioned movably between an edge of an incision and an outer peripheral surface of the retraction ring after said camera extends through the incision.
